(19) United States
(12) Patent Application Publication
(10) Pub. No.: US 2007/0078180 A1
(43) Pub. Date: Apr. 5, 2007

(54) Methods and compositions for stabilizing an antioxidant

(76) Inventors: Dale G. Kern, Hyde Park, UT (US); Thomas M. Schultz, Randolph, NJ (US); Carsten R. Smidt, Sandy, UT (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/243,500

(22) Filed: Oct. 4, 2005

(51) Int. Cl. A61K 31/375; A61K 31/355; A61K 31/122; A61K 31/12; A61K 31/07; A61K 31/015

(52) U.S. Cl. 514/458; 514/474; 514/690; 514/725; 514/763

(57) ABSTRACT

The present invention is a method of stabilizing an antioxidant by adding a carotenoid. According to one embodiment, the method includes stabilizing the antioxidant by reducing oxidation. The method can also include enhancing the beneficial effects of the antioxidant. The antioxidant can be CoQ10. In one aspect of the invention, the carotenoid is a colorless carotenoid.

### FIELD OF THE INVENTION

The present invention relates to a method of stabilizing an antioxidant and further of enhancing the benefits of an antioxidant by adding a carotenoid.

### BACKGROUND OF THE INVENTION

Antioxidants are commonly used in skin care and other health care-related products. One example of an antioxidant used in skin care products is coenzyme Q10, also known as CoQ10 or ubiquinone. A disadvantage associated with antioxidants, including CoQ10, is the instability of the antioxidant during use, especially upon application to the human skin. That is, antioxidants are typically unstable and degrade, thereby losing their antioxidant activity.

There is a need in the art for a method to stabilize antioxidants and enhance their beneficial effects.

### BRIEF SUMMARY OF THE INVENTION

The present invention, in one embodiment, is a method of reducing degradation of an antioxidant. The method includes providing a composition comprising an antioxidant, and adding a carotenoid to the composition, wherein the carotenoid reduces oxidation of the antioxidant. According to one embodiment, the antioxidant is CoQ10. In one alternative aspect of the invention, the carotenoid is a colorless carotenoid.

The present invention, in another embodiment, is a method of prolonging antioxidant activity in a topical skin care composition. The method includes providing a topical skin care composition comprising an antioxidant, and adding a colorless carotenoid to the composition, wherein the antioxidant activity is enhanced by the colorless carotenoid.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a graphical depiction of the effect of UVB radiation on the stability of CoQ10 in PBS, according to one embodiment of the present invention.

FIG. 2 is a graphical depiction of the effect of UVB radiation on the stability of CoQ10 in culture with fibroblasts, according to one embodiment of the present invention.

FIG. 3 is a graphical depiction of the effect of CoQ10 on UV-induced MMP-1 in fibroblasts, according to one embodiment of the present invention.

FIG. 4 is a graphical depiction of the effect of CoQ10 on UV-induced IL-6 in fibroblasts, according to one embodiment of the present invention.

FIG. 5 is a graphical depiction of the effect of CoQ10 on UV-induced PGE-2 in fibroblasts, according to one embodiment of the present invention.

FIG. 6 is a graphical depiction of the effect of CoQ10 on IL-1-induced PGE-2 in fibroblasts, according to one embodiment of the present invention.

FIG. 7 is a graphical depiction of the effect of CoQ10, colorless carotenoids, and a combination of CoQ10 and colorless carotenoids on PGE-2 in fibroblasts, according to one embodiment of the present invention.

FIG. 8 is a graphical depiction of the effect of CoQ10, colorless carotenoids, and a combination of CoQ10 and colorless carotenoids on IL-1-induced MMP-1 in fibroblasts, according to one embodiment of the present invention.

FIG. 9 is a graphical depiction of the effect of lycopene on CoQ10 in the presence of hypochlorite, according to one embodiment of the present invention.

FIG. 10 is a graphical depiction of the effect of lycopene and colorless carotenoids on CoQ10 in the presence of hypochlorite, according to one embodiment of the present invention.

FIG. 11 is a graphical depiction of the effect of colorless carotenoids on CoQ10 in the presence of potassium hydroxide, according to one embodiment of the present invention.

### DETAILED DESCRIPTION

The present invention relates to a method of stabilizing or reducing degradation of an antioxidant by adding a carotenoid. In addition, the present invention relates to a method of stabilizing an antioxidant and enhancing the beneficial effects of the antioxidant by adding a carotenoid. The invention further relates to the resulting compositions.

The present invention, according to one embodiment, is a method of reducing degradation of an antioxidant by adding a carotenoid. In one aspect, the present invention is a method of reducing degradation of an antioxidant in a solution by adding a carotenoid to the solution. In accordance with one embodiment, the carotenoid is a colorless carotenoid, which is available from Israeli Biotechnology Research in Israel. The addition of a carotenoid to an antioxidant, according to one embodiment, has the surprising effect of reducing degradation of the antioxidant. Certain antioxidants are known to be susceptible to oxidation. In one aspect of the invention, it is believed, without being limited by theory, that the addition of the carotenoid reduces the degradation of the antioxidant by reducing oxidation of the antioxidant.

The present invention, according to one embodiment, also includes a method of not only reducing degradation of an antioxidant, but also enhancing the beneficial effects of the antioxidant by adding a carotenoid. The addition of the carotenoid can have the surprising effect of increasing the antioxidizing activity of the antioxidant beyond the expected cumulative benefits of the antioxidant and the carotenoid. That is, according to one embodiment, the beneficial antioxidizing effects of a combination of a antioxidant and a carotenoid exceeds the total benefit of the antioxidant and the carotenoid used separately. In one embodiment, the antioxidant is CoQ10 and the carotenoid is a colorless carotenoid.

The antioxidant useful in the present invention can be coenzyme Q10 (also referred to as “CoQ10” or “ubiquinone”). Alternatively, the antioxidant is another form of CoQ10, such as the reduced form, ubiquinol, or an antioxidant similar to CoQ10, such as, for example, CoQ4, CoQ5, CoQ6, CoQ7, CoQ8, or CoQ9. In a further alternative, the antioxidant is Vitamin E, Vitamin C, Vitamin A, or any other known antioxidant except for a carotenoid, which can exhibit antioxidation characteristics but constitutes a separate component of the present invention as discussed below.

In one aspect of the invention, the carotenoid is a colorless carotenoid. That is, the carotenoid is either phytoene or phytofluene, both of which are available commercially from Israeli Biotechnology Research (“IBR”) in Israel, or some combination of the two. Alternatively, the carotenoid is lycopene. In a further alternative, the carotenoid is any known carotenoid.

According to one embodiment, the antioxidant is in liquid form. For example, CoQ10 can be presented in a liquid form. Alternatively, the antioxidant can be presented in a solid form. For example, according to one embodiment, an antioxidant such as Vitamin A can be presented in a powder form.

In one embodiment, the antioxidant is placed in solution. Alternatively, the antioxidant is presented in a capsule form, such as when it is used as a nutritional supplement. In one aspect of the invention in which the antioxidant is in a solution, the solvent is a lower polarity solvent or a nonpolar solvent such as chloroform or ethanol. For example, in embodiments in which the antioxidant is CoQ10 in solution, the solvent can be a lower polarity or nonpolar solvent. Alternatively, the solvent is a polar solvent such as water. According to one embodiment, the antioxidant is present in the solution in an amount ranging from about 0.0001% by weight to about 99% by weight (also referred to as “wt %”). In an alternative embodiment, 0.001 wt % to about 50 wt %. Alternatively, the antioxidant is present in the solution in an amount ranging from about 0.01 wt % to about 1.0 wt %. In a further alternative, the antioxidant is present in an amount ranging from about 0.01 wt % to about 0.7 wt %.

In accordance with one aspect of the invention, the carotenoid is provided in solution. For example, in one aspect of the invention in which the carotenoid is a colorless carotenoid provided by IBR, the carotenoid is provided in solution in which polydecene is the solvent. Alternatively, the carotenoid can be provided in solution in which the solvent is any lower polarity solvent. In one embodiment, the carotenoid is provided in an amount ranging from about 0.000001 wt % to about 30 wt %. Alternatively, the carotenoid is provided in an amount ranging from about 0.00001 wt % to about 0.01 wt %. In a further alternative, the carotenoid is provided in an amount ranging from about 0.0001 wt % to about 0.1 wt %. In another alternative, the carotenoid is provided in an amount such that the ratio of the amount of carotenoid to the amount of antioxidant ranges from about 1:5 to about 1:2000. In yet a further alternative, the carotenoid is provided in an amount such that the ratio of carotenoid to antioxidant ranges from about 1:10 to about 1:1000. In another alternative embodiment, the carotenoid is provided in an amount such that the ratio of carotenoid to antioxidant ranges from about 1:15 to about 1:100.

The antioxidant and the carotenoid, according to one embodiment, are encapsulated in beads, which are then placed in solution. According to one embodiment, the encapsulation protects the antioxidant and carotenoid from breaking down in solution. For example, the encapsulation could be accomplished using gelatin. Alternatively, the encapsulation could be accomplished using a marine-derived high molecular weight polysaccharide such as an alginate. In a further alternative, liposomes could be used.

According to one embodiment, the addition of a carotenoid to an antioxidant reduces degradation of the antioxidant by reducing oxidation. As discussed above, one problem with antioxidants, including CoQ10, is the instability of the antioxidant during use. Antioxidants are typically unstable in solution and degrade, thereby losing their antioxidant activity. Without being limited by theory, it is believed that the degradation can occur, at least in some instances, as a result of oxidation of the antioxidant. In fact, as can be seen in the Examples herein, CoQ10 is susceptible to oxidation in the presence of sodium hypochlorite, potassium hydroxide, and reactive oxygen species (“ROS”) such as hypochlorous acid, which is a very strong oxidant produced by many cells in the body. ROS are produced by immune cells that are present in the skin at various times. In addition, other free radicals that can cause oxidation of antioxidants are also present in the skin. Thus, the instability of antioxidants, including CoQ10, and the presence of free radicals such as ROS in the skin, can cause the degradation of any composition containing an antioxidant that is intended for skin application, thereby decreasing the effectiveness of the composition.

Without being limited by theory, it is believed that the addition of a carotenoid to the antioxidant inhibits or stops the degradation of the antioxidant by slowing or preventing oxidation of the antioxidant. As is shown in the Examples herein, the addition of various carotenoids to an antioxidant in solution results in the antioxidant resisting oxidation and thereby exhibiting effectiveness as an antioxidant significantly longer than in the absence of the carotenoid.

Thus, addition of a carotenoid stabilizes or prolongs the effectiveness of the antioxidant such that when the antioxidant is applied in any form to the human skin, free radicals and other oxidating entities present in the skin are inhibited from causing oxidation of the antioxidant. As such, the antioxidant remains active longer and, according to one embodiment, can even remain active while travelling through the stratum corneum to epidermal and dermal cells.

The present invention, according to one embodiment, is a composition comprising an antioxidant and a carotenoid. According to one embodiment, the antioxidant is present in an amount ranging from about 0.001% by weight to about 50% by weight (also referred to as “wt %”). Alternatively, the antioxidant is present in the solution in an amount ranging from about 0.01 wt % to about 1.0 wt %. In a further alternative, the antioxidant is present in an amount ranging from about 0.1 wt % to about 0.7 wt %. In one embodiment, the carotenoid is present in an amount ranging from about 0.000001 wt % to about 0.01% wt %. Alternatively, the carotenoid is present in an amount ranging from about 0.00001 wt % to about 0.0001 wt %. In a further alternative, the carotenoid is present in an amount such that the ratio of the amount of carotenoid to the amount of antioxidant ranges from about 1:10 to about 1:30. In yet a further alternative, the carotenoid is provided in an amount such that the ratio of carotenoid to antioxidant ranges from about 1:15 to about 1:25. In another alternative embodiment, the carotenoid is provided in an amount such that the ratio of carotenoid to antioxidant ranges from about 1:18 to about 1:22.

In one aspect of the invention, the composition is a topical cream further comprising an appropriate carrier. The composition, according to one embodiment, comprises water, an antioxidant, and a carotenoid. Alternatively, the composition can also include at least one solubilizer, at least one thickening agent, at least one additional antioxidant, at least one additional carotenoid, and/or an anti-inflammation agent. A solubilizer, according to one embodiment, is any component that allows the incorporation of hydrophobic ingredients in the composition. In one example, the solubilizer is polyoxyethylene castor oil. In one embodiment, an example of a thickening agent is hydroxyethylcellulose.

The composition can also include a surfactant/emulsifying agent, such as PEG-60 Hydrogenated Castor Oil, Polyoxyethylene Castor Oil, Glycine Soya, Ceteareth-12, Ceteareth-20, or Glyceryl Stearate. In addition, the composition can also include an emulsion stabilizer, such as Cetearyl Alcohol, Acrylates/C10-30 Alkyl Acrylate Crosspolymer, or a cellulose. In a further embodiment, the composition can include a humectant (brings atmospheric water to the skin surface upon application), such as Glycerin, diglycerin, lactose, or mannitol. Further, the composition can include an emollient (which softens the skin) such as raffinose or glyceryl stearate. In yet another embodiment, the composition can include a microbiological preserving agent such as Phenoxyethanol, methyparaben, or propylparaben. In a further alternative, the composition can also include an antioxidant such as Tocopheryl acetate (form of Vitamin E), ascorbic acid, or ascorbyl palmitate (form of Vitamin C).

The composition according to one embodiment is a water-based composition. A water-based composition is most effective when most or all of the ingredients are water soluble, are solubilized with surfactants/emulsifying agents, or are encapsulated as described herein elsewhere.

Alternatively, the composition is an emulsion based on a water-in-oil or oil-in-water formulation. Such a composition is most effective when it contains more hydrophobic ingredients, either due to the active agents to be delivered to the skin or because the organoleptic properties desired. The most common is the oil-in-water. Emulsifying agents are used to create a simple emulsion where the hydrophobic ingredients are “protected” in micelles that then are found distributed within the main water phase. Ingredients are placed in the micelles and in the water phase to stabilize the micelles.

The water-in-oil system is a bit more complicated and the ingredients must be more carefully chosen. Water-in-oil emulsions often provide unique feel and “break” (that is the way the product rubs into the skin) compared to the oil-in-water formulations. Typically, water-in-oil emulsions contain several emulsifying agents and emulsion-stabilizing agents.

Alternatively, the composition is a capsule. For example, according to one embodiment, the antioxidant can be dissolved in a solution of carotenoid and the resulting composition encapsulated in a soft gel capsule. In use, according to one embodiment, the capsule could be broken open and the contents rubbed directly on the skin.

Alternatively, the composition is any solution or dispersion appropriate for application to the human skin that contains an antioxidant and a carotenoid as described herein.

The composition, according to one embodiment, can exhibit anti-inflammatory or anti-aging properties when applied to human skin. It is known that certain antioxidants exhibit anti-inflammatory and anti-aging properties. Colorless carotenoids exhibit anti-inflammatory properties as well. More specifically, colorless carotenoids exhibit a direct inhibitory effect on the production of inflammatory mediators in skin cells. Thus, the combination of an antioxidant such as CoQ10 and a carotenoid in a composition exhibits anti-inflammatory and anti-aging properties. In fact, as discussed above and shown in the Examples, the combination of an antioxidant and a carotenoid can have synergistic anti-inflammation and anti-aging effects. That is, in one aspect of the invention, a combination of an antioxidant and a carotenoid produce an enhanced inhibition of free radicals in human skin.

CoQ10 or ubiquinone is a coenzyme, which is a cofactor upon which the comparatively large and complex enzymes depend for their function. CoQ10 is the coenzyme for at least three mitochondrial enzymes as well as enzymes in other parts of the cell. In the mitochondria, CoQ10 is a vital component of the oxidative phosphorylation pathway that produces adenosine triphosphate (ATP), upon which all cellular functions depend for energy.

Antioxidants, including CoQ10, exhibit anti-inflammatory and anti-aging properties. For example, as shown in the Examples herein, CoQ10 can lower levels of inflammatory mediators such as IL-6 and PGE-2, thereby reducing inflammation in a patient. Further, CoQ10 has been shown to lower levels of collagenase activity, which has been associated with such signs of aging as wrinkles. Without being limited by theory, it is believed that at least a portion of an antioxidant's anti-inflammatory and anti-aging activity, including that of CoQ10, may result from its ability to neutralize free radicals. For example, it is likely that the free radical neutralization capabilities arise from CoQ10's ability to accept and donate electrons. The ability of CoQ10 to donate electrons in its reduced state makes CoQ10 an excellent antioxidant, being able to transfer electrons to free radicals such as the hydroxyl radical. Further, CoQ10 in its oxidized state can accept electrons from free radicals such as the superoxide radical.

Although the present invention has been described with reference to preferred embodiments, persons skilled in the art will recognize that changes may be made in form and detail without departing from the spirit and scope of the invention.

### EXAMPLES

The following examples are presented by way of demonstration, and not limitation, of the invention.

Unless otherwise indicated, the following procedures were employed for the following examples:

HPLC Methods. To measure CoQ10 levels in tissue culture medium, an appropriate high performance liquid chromatography (“HPLC”) method had to be established.

HPLC is used to identify the amount of a chemical compound within a mixture of other chemicals. In operation, the sample to be tested is dissolved in a solvent (like water or alcohol) and pumped through an apparatus that characterizes the components of the sample. The HPLC method operates by comparing the characteristics of the solvent alone and the characteristics of the solvent AND the sample.

For the instant examples, a reliable HPLC method was established for CoQ10 using an isochratic method with a mobile phase of 85% methanol and 15% hexane. CoQ10 was detected at 275 nm. Standard curves were developed and the limit of detection of CoQ10 was found to be 100 ng/ml.

HPLC Analysis of CoQ10. The amount of CoQ10 remaining in degradation experiments below were determined by High Pressure Liquid Chromatography (HPLC). The column used was a Microsorb C18 reverse phase column (15 cm×0.46 cm), the mobile phase consisted of 85% methanol and 15% hexane at a flow rate of 1 ml/minute and CoQ10 was detected at 235 nm. The instrument used was a HP 1090 gradient HPLC system with photodiode array detector and autosampler. Standard curves had a limit of detection for CoQ10 of 100 ng/ml.

Mixtures of Colorless Carotenoids. The colorless carotenoids used in the following Examples were obtained from Israeli Biotechnology Research in Tel Aviv, Israel and were supplied as either a DMSO solution of a mixture of phytoene and phytofluene with a total concentration of 1.5 milligrams/ml or as a polydecene oil solution containing 0.07% phytoene and 0.006% phytofluene.

### Example 1

Methods and Materials. The following experiment involved examining the effect of ultraviolet radiation (“UVR”) on levels of CoQ10 in culture medium. More specifically, ultraviolet-B (“UVB,” which is ultraviolet radiation ranging from about 290 nm to about 320 nm) dose-response studies were conducted on various concentrations of CoQ10 placed in either PBS buffer or into culture media. Doses of UVB ranged from 50 mJ to 200 mJ. In the culture media, 10 μm of CoQ10 was placed in a fibroblast culture for 24 hours.

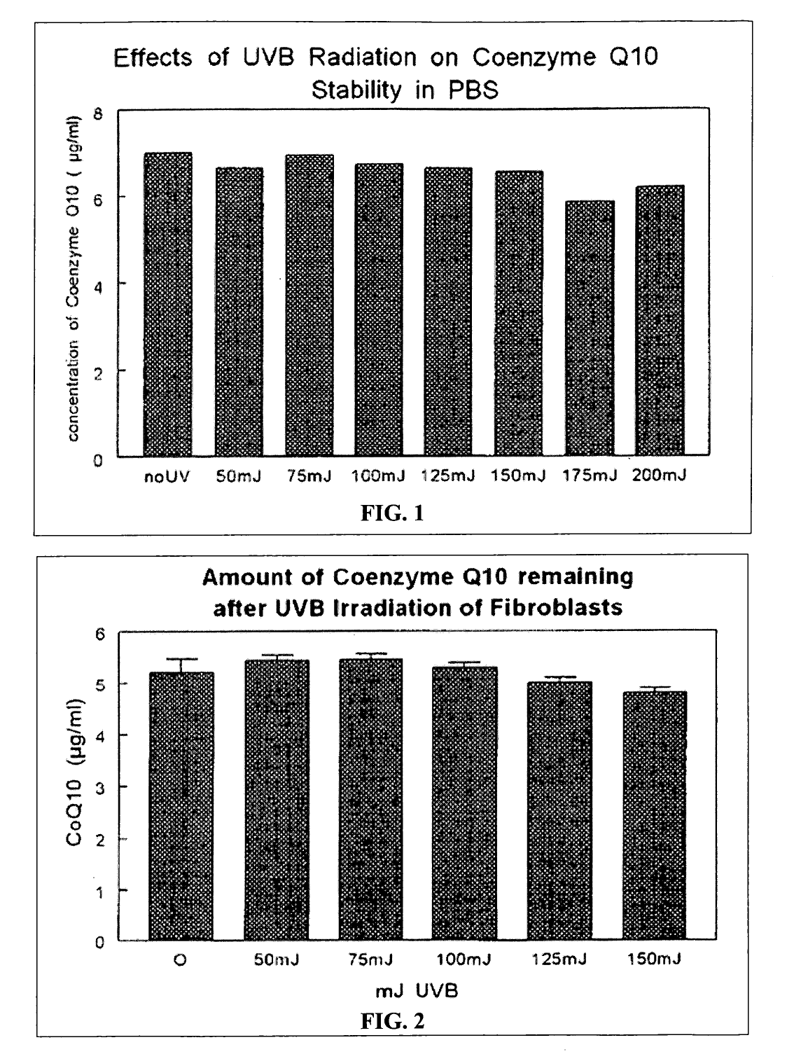

Results. As shown in FIG. 1, CoQ10 in PBS buffer exhibited stability to all UVB doses ranging from 50 mJ to 200 mJ. The small (<10) decrease in the concentration of CoQ10 at high UVB doses was within experimental error.

As shown in FIG. 2, the amount of CoQ10 in a fibroblast culture was generally unaffected by the UVB radiation, exhibiting only a slight reduction.

Discussion. Thus, CoQ10 appears to be stable when exposed to UVR, at least up to 200 mJ. Further, it appears that CoQ10 is stable at 37° C. in tissue culture medium in the presence of irradiated dermal fibroblasts. Thus, any free radicals that might have been generated by UVR treated cells had no impact on CoQ10 levels, likely because CoQ10 can exist in a stable form in either the oxidize or reduced state. Further, with respect to the culture medium, since tissue culture medium contains a variety of vitamins, fatty acids, and other compounds that have antioxidant activity, it is likely that any free radicals generated by the irradiated cells were trapped by the antioxidants in the culture medium.

### Example 2

Methods and Materials. The following experiment involved examining the effect of CoQ10 on UVR-induced inflammatory mediators in human dermal fibroblasts. That is, because skin aging is known to be accelerated by inflammatory cytokines produced in the skin by UVR, this experiment assessed the ability of CoQ10 to inhibit the production of the inflammatory mediators produced in human dermal fibroblasts in response to either UVB radiation or to stimulation by IL-1, a pro-inflammatory cytokine produced in keratinocytes by UVR.

The dose of UV for stimulating the production in fibroblasts of each of the inflammatory mediators—PGE-2, IL-6, IL-8, and the matrix metalloproteinase (“MMP-1”)—was 50 mJ. This was determined to be the optimum dose for stimulating the production of the mediators because it produced a good induction of inflammatory mediators but did not lower the growth or survival of the cells.

The cells were isolated and cultured as follows. The dermal fibroblasts were isolated from human neonatal foreskin and cultured in Dulbecco's modified eagle media with L-glutamine and 4.5 mM glucose and without sodium pyruvate (“DMEM”) (available from Fisher Scientific, Pittsburgh, Pa.) containing 5% horse serum (available from Hyclone, Logan, Utah), 5% fetal bovine serum (available from Hyclone), and penicillin/streptomycin (100 U/100 μg/ml). Cell cultures were grown at 37° C. in a humidified CO₂ incubator (5% CO₂). Cells were passaged prior to confluence by removing cells with a trypsin/EDTA solution, followed by centrifugation and re-seeding.

For the experiments, cells were seeded into 12 well culture dishes in complete growth medium at a density of 10⁵ cells/well and allowed to attach overnight. The medium was then replaced with PBS for irradiation. A bank of two commercially available FS20 lamps were used for irradiation of cells. The lamps were mounted 35 cm above the culture dishes such that the radiation reaching the cells beneath the culture dish cover was 50 μWatts of UV and 35 μwatts of UVA. After irradiation with 50 mJ as explained above, the PBS was replaced with growth medium containing the appropriate concentration of test compounds. Cells were incubated for 24 hours at 37° C., at which time the media was removed for assay and the cells counted. The values provided in the results are the averages of six determinations of number of cells±standard deviation. The experiment for each mediator was repeated twice with similar results.

Because of the very poor solubility of CoQ10 in any solvent that is compatible with cultured cells, 10 μm of CoQ10 was the maximum final concentration that could be used in culture medium.

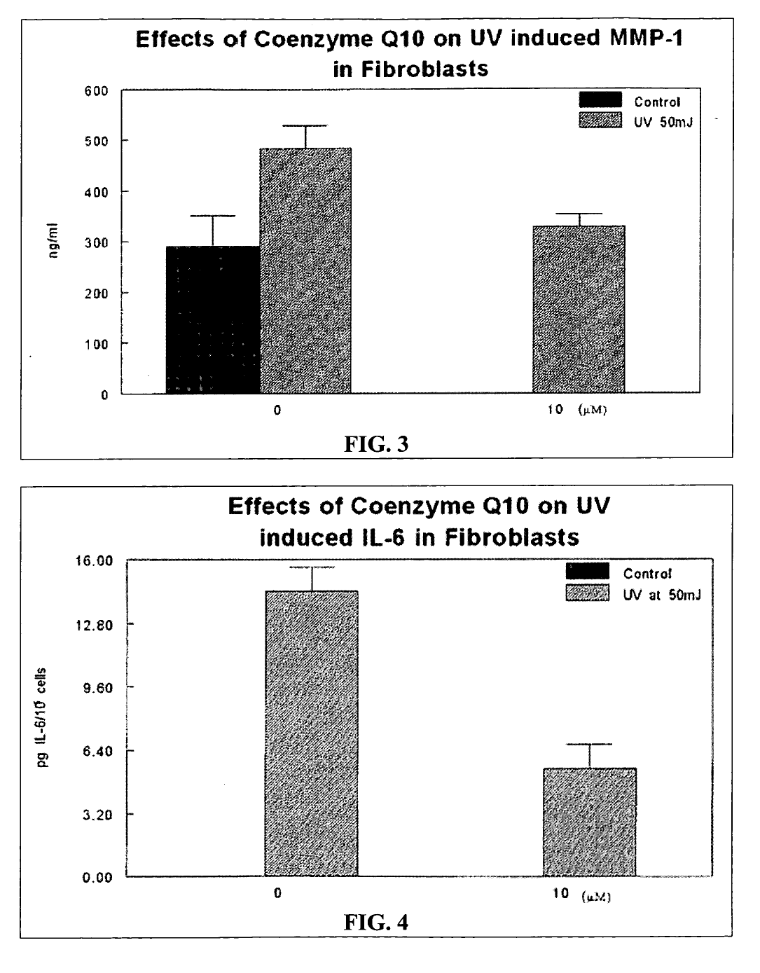

One inflammatory mediator examined was MMP-1. The MMP-1 kits were obtained from R&D Systems in Minneapolis, Minn. Two plates of cells were prepared as described above. After irradiation of both plates to stimulate production of MMP-1, 10 μM of CoQ10 in DMSO was added to one of the plates and both plates were incubated for 24 hours and then the media was removed from culture and assayed for MMP-1 with the use of a commercially-available ELISA kit. The results are shown in FIG. 3.

Another inflammatory mediator examined was cytokine IL-6. The IL-6 kit was obtained from R&D Systems in Minneapolis, Minn. Two plates of cells were prepared as described above. After irradiation of both plates to stimulate production of IL-6, 10 μM of CoQ10 in DMSO was added to one of the plates and both plates were incubated for 24 hours and then the media was removed from culture and assayed for IL-6 with the use of a commercially-available ELISA kit. The results are shown in FIG. 4.

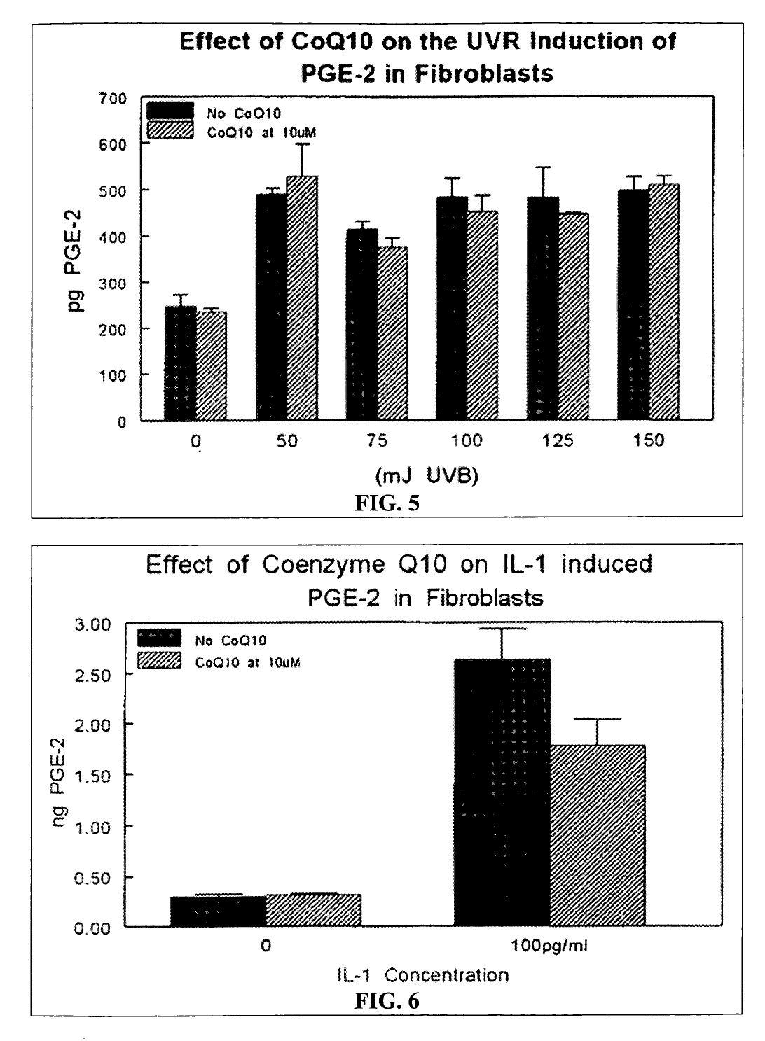

A further mediator examined was PGE-2. ELISA kits for PGE-2 were obtained from Amersham Biosciences in Piscataway, N.J. Two plates of cells were prepared as described above. After irradiation of both plates to stimulate production of PGE-2, 10 μM of CoQ10 in DMSO was added to one of the plates and both plates were incubated for 24 hours and then the media was removed from culture and assayed for PGE-2 with the use of a commercially-available ELISA kit. The results are shown in FIG. 5.

PGE-2 in fibroblasts stimulated with IL-1 was also examined. IL-1 was obtained from Sigma Chemical in St. Louis, Mo. Two plates of cells were prepared and allowed to attach overnight as described above. The medium was then replaced with fresh medium containing 100 picograms/ml of IL-1. After irradiation of both plates to stimulate production of PGE-2, 10 μM of CoQ10 in DMSO was added to one of the plates and both plates were incubated for 24 hours and then the media was removed from culture and assayed for PGE-2 with the use of a commercially-available ELISA kit. The results are shown in FIG. 6.

Results. As shown in FIG. 3, CoQ10 at a concentration of only 10 micromolar was effective in reducing the level of UVR-induced MMP1 back to near control levels.

As shown in FIG. 4, CoQ10, at a concentration of 10 micromolar, markedly lowered the level of the inflammatory cytokine IL-6 induced in UVR-treated fibroblasts.

As shown in FIG. 5, CoQ10 had only a small inhibitory effect on levels of PGE-2 at UVB irradiation doses of 75 mJ, 100 mJ, and 125 mJ.

However, as shown in FIG. 6, when the fibroblasts were stimulated with IL-1 (a pro-inflammatory cytokine produced in the skin in response to UV irradiation which up-regulates many cytokines as well as PGE-2 and MMPs), CoQ10 at a concentration of 10 micromolar caused a significant reduction in the IL-1 induction of PGE-2.

Discussion. These results suggest that CoQ10 does, in fact, have some beneficial effects on skin cells, including lowering the inflammatory state of the skin and protecting the skin against UVR-induced aging effects, particularly by blocking collagenase-mediated destruction of collagen in the dermal matrix.

### Example 3

Methods and Materials. The following experiment involved examining the effect of the colorless carotenoids (phytoene and phytofluene) on inflammatory mediator production and on CoQ10 biological activity.

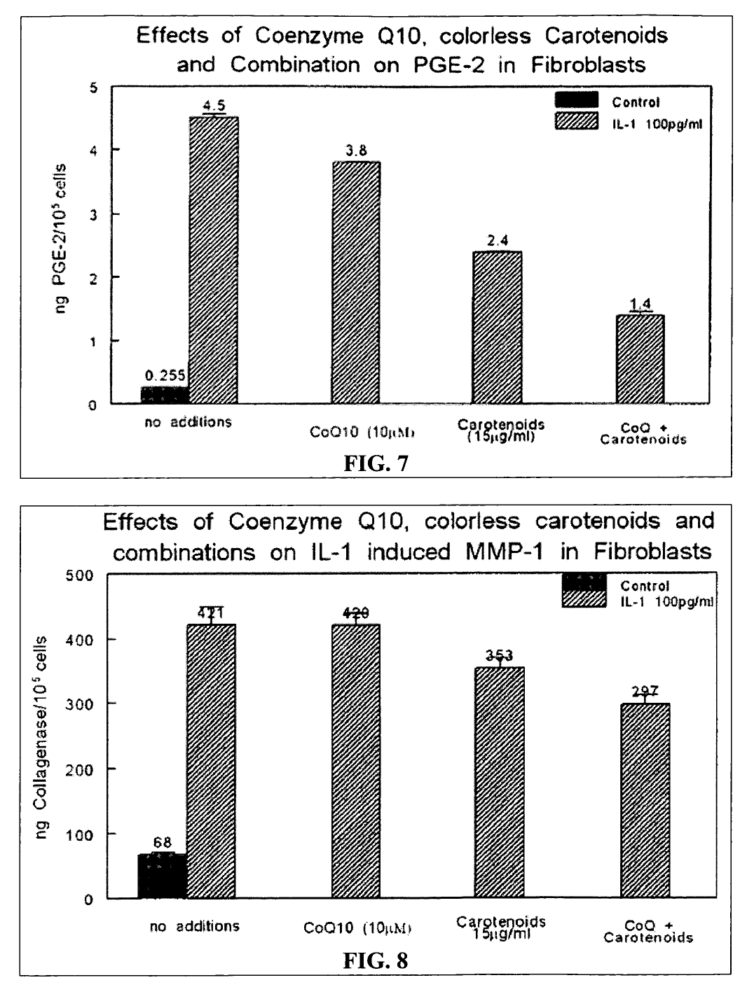

The impact of CoQ10, colorless carotenoids, and a combination of both on PGE-2 in fibroblasts stimulated with IL-1 was examined. IL-1 was obtained from Sigma Chemical in St. Louis, Mo. Eight plates of cells were prepared and allowed to attach overnight as described above. The medium was then replaced with fresh medium, and in half the plates (four of the plates), the fresh medium contained IL-1 (making them IL-1+). The other four plates were the control plates with respect to IL-1 (making them IL-1−). After irradiation of all eight plates to stimulate production of PGE-2, 10 μM of CoQ10 in DMSO was added to two of the IL-1 plates (making them IL-1+, CoQ10+) and two of the control plates (making them IL-1−, CoQ10+). Further, 15 micrograms/ml of the colorless carotenoid was added to the IL-1−, CoQ10+ plate (making it IL-1−, CoQ10+, carot+), to the IL-1−, CoQ10− plate (making it IL-1−, CoQ10−, carot+), to the IL-1+, CoQ10+ plate (making it IL-1+, CoQ10+, carot+), and to the IL-1+, CoQ10− plate (making it IL-1+, CoQ10−, carot+). All of the plates were then incubated for 24 hours and then the media was removed from culture and cells counted. The results are shown in FIG. 7.

The impact of CoQ10, colorless carotenoids, and a combination of both on MMP-1 in fibroblasts stimulated with IL-1 was examined. IL-1 was obtained from Sigma Chemical in St. Louis, Mo. Eight plates of cells were prepared and allowed to attach overnight as described above. The medium was then replaced with fresh medium, and in half the plates (four of the plates), the fresh medium contained IL-1 (making them IL-1+). The other four plates were the control plates with respect to IL-1 (making them IL-1−). After irradiation of all eight plates to stimulate production of MMP-1, 10 μM of CoQ10 in DMSO was added to two of the IL-1 plates (making them IL-1+, CoQ10+) and two of the control plates (making them IL-1−, CoQ10+). Further, 15 micrograms/ml of the colorless carotenoid was added to the IL-1−, CoQ10+ plate (making it IL-1−, CoQ10+, carot+), to the IL-1−, CoQ10− plate (making it IL-1−, CoQ10−, carot+), to the IL-1+, CoQ10+ plate (making it IL-1+, CoQ10+, carot+), and to the IL-1+, CoQ10− plate (making it IL-1+, CoQ10−, carot+). All of the plates were then incubated for 24 hours and then the media was removed from culture and assayed for MMP-1 with the use of a commercially-available ELISA kit. The results are shown in FIG. 8.

Results. The combination of CoQ10 and the colorless carotenoids produced either an additive or synergistic inhibitory effect on the production of PGE-2 and MMP-1.

As shown in FIG. 7, CoQ10 alone caused a 16% inhibition of the IL-1 induction of PGE-2. The carotenoids alone inhibited the IL-1 increase in PGE-2 by a very significant 47%. The combination of CoQ10 and carotenoids produced an almost 70% inhibition of PGE-2 production.

As shown in FIG. 8, CoQ10 was ineffective in blocking the IL-1 induction in MMP-1 (recall that CoQ10 can reduce MMP-1 in UVR-treated cells, but may not be able to block the strong induction of MMP-1 by the potent IL-1). The colorless carotenoids, however, were able to cause a small inhibition of approximately 16% of IL-1 induction of MMP-1. Interestingly, the combination of CoQ10 and the colorless carotenoids produced an almost 30% decrease in MMP-1.

Discussion. The results shown in FIG. 7 suggest that CoQ10 and colorless carotenoids at least work additively to lower PGE-2 and may actually work synergistically since an additive effect would result in a 63% inhibition of PGE-2, and not the 70% level of inhibition that was found. The results shown in FIG. 8 indicate that the combination of CoQ10 and carotenoids create a pronounced synergistic inhibitory effect.

### Example 4

Methods and Materials. The following experiment examined the effect of the hydroxyl radical on CoQ10. The hydroxyl radical is a reactive oxygen species (“ROS”) produced in the human body. The radical is generated by the reaction of iron with hydrogen peroxide (commonly called the Fenton reaction).

For this experiment, CoQ10 was prepared in a solution of chloroform/ethanol (1:1) at a concentration of 10 micrograms/ml. The Fenton reaction was then initiated by the addition of hydrogen peroxide and iron (III) chloride and allowed to proceed at room temperature for one hour, at which time samples were injected on HPLC.

Results. The generation of hydroxyl radicals was verified using the carotenoid lycopene as a target for hydroxyl radicals. When the above Fenton reaction was performed on lycopene, the amount of lycopene was reduced by 50%, indicating that hydroxyl radicals were in fact being generated. However, the generation of hydroxyl radicals by the Fenton reaction did not cause a reduction in the abundance or a change in structure of CoQ10 as assessed by HPLC.

Discussion. Thus, the experiment indicates that CoQ10 is not degraded by hydroxyl radicals.

### Example 5

Methods and Materials. The following experiment examined the effect of sodium hypochlorite on CoQ10. Sodium hypochlorite converts to hypochlorous acid, the most active ROS produced by cells.

For this experiment, CoQ10 was prepared in HPLC “mobil phase” (methanol:hexane) and incubated with different concentrations of sodium hypochlorite prepared in water/methanol. The reaction was allowed to proceed for up to 30 minutes, at which time samples were injected on HPLC.

Results. As shown in FIG. 9, sodium hypochlorite caused a 50% loss of CoQ10 as detected by HPLC.

Discussion. Thus, the experiment indicates that CoQ10 is degraded by hypochlorous acid.

### Example 6

Methods and Materials. The following experiment examined whether a commercially available lycopene could protect CoQ10 from degradation.

The stock lycopene was 10 mg/ml in soybean oil. It was diluted into toluene and used in the reaction at 0.1% lycopene. To allow the CoQ10, hypochlorite aqueous solution and lycopene to react, a detergent was used to bring the polar and nonpolar phases together. The incubation lasted for 30 minutes.

Results. As shown in FIG. 9 above, the oxidation of CoQ10 by hypochlorite was significantly reduced when lycopene was incubated with CoQ10, indicating that the carotenoid could protect CoQ10 from oxidation.

Discussion. The role of carotenoids in protecting proteins, fatty acids, and small compounds is well known and their role in protecting LDL from oxidation by hypochlorous acid/hypochlorite produced by many cells in the body has been the focus of considerable research. Studies have shown that the oxidation of LDLs can be suppressed by carotenoids which bind to LDL and scavenge the hypochlorite. Of the carotenoids known to protect LDL against hypochlorite oxidation, the most effective is lycopene. Thus, the above results suggest that, like their role in LDL protection, carotenoids, and particularly lycopene, can protect CoQ10 from oxidation.

### Example 7

Methods and Materials. The following experiment was a comparison of the impact that colorless carotenoids or lycopene have on CoQ10 when CoQ10 is exposed to hypochlorite oxidation.

695 μg/ml of CoQ10 was prepared in chloroform/ethanol (1:1). The calculated amounts of phytoene and phytofluene used in the study was 0.0142% and 0.0012%, respectively. The calculated amount of lycopene was 2 mg/ml in chloroform. Four plates of cells were prepared and allowed to attach overnight as described above. CoQ10 was then added to each of the plates. Further, lycopene was added to one of the plates, and the carotenoids were added to another. Three plates (one of the plates containing only CoQ10 was treated as a control) were then incubated with 0.03% hypochlorite (“HOCL”) for 1.5 hours. Then the samples were removed and assayed by HPLC to determine CoQ10 levels.

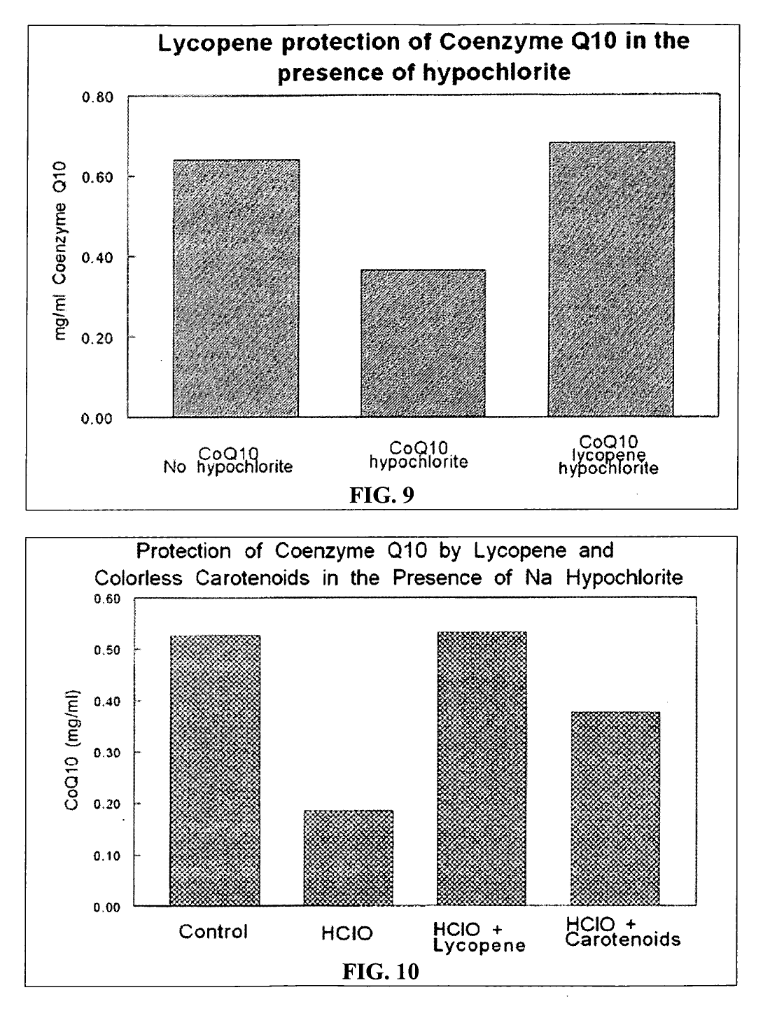

Results. As shown in FIG. 10, hypochlorite caused a 60% loss of CoQ10 as determined by HPLC. As shown above, the addition of lycopene caused a reduction in oxidation of CoQ10 by hypochlorite. When colorless carotenoids were used, the oxidation of CoQ10 by hypochlorite was also reduced. This experiment was repeated three times with similar results.

### Example 8

Methods and Materials. The following experiment examined whether colorless carotenoids could protect CoQ10 from potassium hydroxide oxidation.

600 μM of CoQ10 was prepared in chloroform stock. 500 μg/ml of the colorless carotenoids was prepared in DMSO. The calculated amount of potassium hydroxide (“KOH”) was 1 mM. Three plates of cells were prepared and allowed to attach overnight as described above. CoQ10 was then added to each of the plates. Further, KOH was added to two of the plates (one was maintained as a control containing only CoQ10). Further, the carotenoids were added to one of the plates containing KOH. Reactions were performed in a total volume of 1 ml. The cells containing KOH were incubated for 30 minutes at room temperature with the KOH. Then the samples were removed and assayed by HPLC to determine CoQ10 levels.

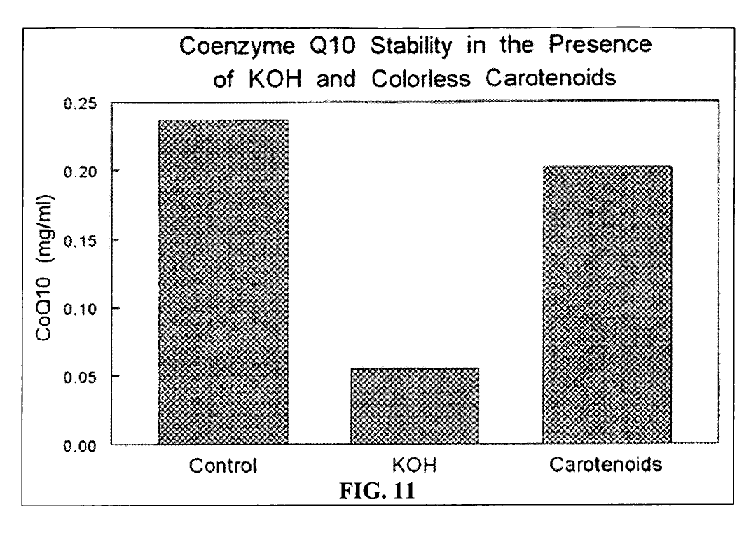

Results. As shown in FIG. 11, potassium hydroxide caused an 80% loss of CoQ10 due to oxidation as detected by HPLC. The addition of the carotenoids prevented the oxidation of CoQ10 by potassium hydroxide. This experiment was repeated three times with similar results.

Discussion. These results provide further evidence that phytoene and phytofluene work to protect CoQ10 from degradation by oxidation.

What is claimed is:

1. A method of reducing degradation of an antioxidant, the method comprising:
providing a composition comprising an antioxidant; and
adding a carotenoid to the composition, wherein the carotenoid reduces oxidation of the antioxidant.

2. The method of claim 1, wherein the composition is a solution.

3. The method of claim 1, wherein the antioxidant is present in the composition in an amount ranging from about 0.0001 wt % to about 50 wt %.

4. The method of claim 1, wherein the carotenoid is in solution.

5. The method of claim 1, wherein the carotenoid is added to the composition in an amount ranging from about 0.0001 wt % to about 0.1 wt %.

6. The method of claim 1, wherein the carotenoid is a colorless carotenoid.

7. The method of claim 5 wherein the colorless carotenoid is phytoene.

8. The method of claim 5 wherein the colorless carotenoid is phytofluene.

9. The method of claim 1 wherein the carotenoid is lycopene.

10. The method of claim 1 wherein the antioxidant is ubiquinone.

11. The method of claim 1 wherein the antioxidant is vitamin E.

12. The method of claim 1 wherein the antioxidant is vitamin C.

13. The method of claim 1 wherein the antioxidant is vitamin A.

14. A method of prolonging antioxidant activity in a topical skin care composition, the method comprising:
providing a topical skin care composition comprising an antioxidant; and
adding a colorless carotenoid to the composition, wherein the antioxidant activity is enhanced by the colorless carotenoid.

15. The method of claim 14, wherein the antioxidant is present in the composition in an amount ranging from about 0.0001 wt % to about 50 wt %.

16. The method of claim 14, wherein the antioxidant is present in the composition in an amount ranging from about 0.001 wt % to about 1.0 wt %.

17. The method of claim 14, wherein the carotenoid is added to the composition in an amount ranging from about 0.0001 wt % to about 0.1 wt %.

18. The method of claim 14, wherein the carotenoid is added to the composition in an amount ranging from about 0.000001 wt % to about 0.01 wt %.

19. The method of claim 14, wherein the carotenoid is chosen from a group consisting of phytoene, phytofluene, and lycopene.

20. The method of claim 1 wherein the antioxidant is chosen from a group consisting of ubiquinone, vitamin E, vitamin C, vitamin A.
